(19) United States
(12) Patent Application Publication
(10) Pub. No.: US 2008/0014145 A1
(43) Pub. Date: Jan. 17, 2008

(54) ABALONE SETTLEMENT BIOASSAY FOR ANTI-SEIZURE SUBSTANCE SCREENING

(76) Inventor: David Joseph Anschel, Rocky Point, NY (US)

Correspondence Address: DAVID JOSEPH ANSCHEL, 1 WILDWOOD ROAD, ROCKY POINT, NY, 11778, US

(21) Appl. No.: 11/456,837

(22) Filed: Jul. 11, 2006

(51) Int. Cl. A61K 49/00

(52) U.S. Cl. 424/9.2

(57) ABSTRACT

The present invention describes a method for utilizing marine mollusks in a novel assay for screening candidate substances for anti-epileptic/anti-seizure/anti-convulsant activity. The purpose of the invention is to facilitate the discovery of new treatments for neurological and mental diseases. In particular the use of a larval settlement assay using the genus Haliotis is detailed. As a natural extension of the current invention is the use of known abalone settlement inducers as novel anti-convulsant.

### FIELD OF THE INVENTION

The present invention relates to the utilization of marine organisms of the genus Haliotis in a novel assay for the screening of anti-epileptic (anti-seizure, anti-convulsant) compounds.

### BACKGROUND OF THE INVENTION

Epilepsy is the most prevalent neurological illness which affects people of all ages. Approximately one third of all cases (˜1,000,000 people in USA) are refractory to current medications. New treatments are urgently needed. Anti-epileptic drug (AED) screening depends largely upon animal models of epilepsy. Most of these assays involve rats, with other mammals utilized to a lesser extent. These assays do not perfectly model human epilepsy. Additionally, they are costly, time consuming, and labor intensive. There is a need for better assays to facilitate the discovery of AEDs. The present proposal involves the development of a novel assay using a marine mollusk which can potentially address the above shortcomings of currently employed techniques.

This invention utilizes marine organisms of the genus Haliotis in a novel assay for the screening of anti-epileptic (anti-seizure, anti-convulsant) compounds.

Marine gastropod molluscs of the genus Haliotis occur throughout the world. Commonly known as abalone, they are an important food source and have been extensively studied in order to better understand their maturation and development. In particular, the larval development of Haliotis rufescens (red abalone) has been the subject of much research. After fertilization the larvae are free floating. Eventually (usually after several days) they must stop swimming, sink to the bottom and attach to a substrate in order to begin the process of metamorphosis which will eventually result in the mature phenotype. This process has been termed “settlement”. It was originally discovered by Morse et al. (1979) that settlement can be induced by a chemical cue. His pioneering study utilized γ-aminobutyric acid (GABA) for this purpose. Since then, over the past 25 years many carefully performed experiments, by researchers from all over the world, have demonstrated that in addition to GABA, several other substances may promote or inhibit Haliotis larvae settlement. Much of this progress was reviewed in 2001 by Roberts.

The biochemistry of Haliotis (primarily Haliotis rufescens) settlement is strikingly similar to that of epilepsy. The first (and most widely researched) substance discovered to have the ability to induce Haliotis settlement was GABA. GABA is also the principle inhibitory transmitter in the human brain. A concentration of 10⁻⁶ molar GABA in sea water will induce 96-100% larval attachment in Haliotis rufescens (Morse 1979 and 1980). In the absence of GABA or another an inducer, there will be no settlement or larval development. This result has been replicated by numerous other groups for several species of Haliotis (Slattery 1992, Searcy-Bernal 1992, Barlow 1990 and others). Numerous substances with molecular structures similar to GABA can also induce Haliotis larvae settlement (although generally less efficiently). Some examples include muscimol (Trapido-Rosenthal 1985), L-glutamate, β-alanine, glycine, δ-amniovaleric, ε-amniocaproic acid, and cyclic analogs of GABA (Morse 1980). Although L-glutamate did slowly promote larval settlement D-glutamate and L-glutamine were completely ineffective. It has been proposed that this is due to the fact that L-glutamate may be enzymatically decarboxylated to yield GABA. While D-glutamate and L-glutamine are not substrates for L-glutamic acid decarboxylase (Morse 1980).

GABA and it's receptors are also widely believed to play a key role in the pathophysiology of epilepsy. The following anti-epileptic medications are thought to treat seizures by potentiating GABA_{A}—mediated responses or increasing brain synaptic GABA levels—benzodiazepines, phenobarbital, felbamate, lamotrigine, levetiracetam, tiagabine, and vigabatrin. In addition several others are suspected to at least partially involve these mechanisms (Perucca 2005). According to the United States National Institutes of Health website http://clinicaltrials.gov/ct/gui/show/NCT00005925?order=1 there is even an ongoing human clinical trial which involves directly injecting the potent GABA agonist, muscimol directly into the brains of people with epilepsy. In addition, blockade of GABA receptors using drugs such as bicuculline, pentylenetetrazole or picrotoxin is a common method used to induce seizures in animals (Fisher 1989).

Increasing external potassium has been shown to induce Haliotis rufenscens larva settlement. Baloun et al (1984) demonstrated that potassium added as either a sulfate or chloride salt and as a replacement for either Na⁺ or Mg⁺² could induce larval settlement in a dose dependent manner. At a concentration of ˜10 mM above that found in seawater, up to 90% of Haliotis rufenscens larva will attach within 72 hours (Baloun 1984, Yool 1986). Additionally, a low concentration of K⁺ will inhibit induction by GABA. At higher concentrations K⁺ will not promote settlement. There is therefore a potassium concentration “window” for inducing larval settlement (Yool et al. 1986).

It has been postulated that potassium may play a role in the termination of hippocampal epileptic discharges (Bragin 1997). Several potassium channel genes have been implicated in either epilepsy or resistance to seizures (for example see Lenzen 2005, and Wallace 2004). Additionally, the focal perfusion of local areas of rat cerebral cortex with potassium chloride may prevent or abort chemically induced electrographic seizures (personal experience). Under certain conditions, extracellular potassium may also have pro-epileptogenic effects (Bihi 2005). Potassium may then be either inhibitory or facilitatory of both abalone larval settlement and seizures depending upon the concentration and other variables.

Various alterations of the external Ca²⁺ concentration have different effects upon Haliotis rufescens larval settlement. The most marked is that very low external Ca²⁺ concentration result in the inhibition of larval settlement in multiple conditions including the presence of 4×10⁻⁷ M GABA (Baloun 1984). Decreased extracellular free Ca²⁺ may also provoke epileptic discharges from brain tissue. This effect is thought to be mediated by voltage-dependent Ca²⁺ channels (VDCCs). In addition a Ca²⁺ dependent after hyperpolarization has been hypothesized to play a role in seizure termination. The role of VDCCs in epilepsy is strongly supported by several genetic animal models of epilepsy which have VDCC associated mutations. Some VDCC subunit genes have also been implicated in human epilepsy. Further several anti-epileptic medications apparently act by targeting VDCCs. The role of Ca²⁺ in epilepsy has recently been reviewed in detail (Jones 2002).

In addition to GABA, induction has been triggered in the larvae of Haliotis discus hannai (Japanese abalone) using bromomethane (Kang 2004). Baloun and Morse (1984) have also discovered that settlement of Haliotis rufescens larvae may be induced by replacing 25-75% of the Cl⁻ in seawater with either Br⁻ or one of several other anions. Amazingly bromine compounds are also one of the oldest known pharmacotherapy's for epilepsy. They are rarely still used in humans since the discovery of newer medications with fewer side effects. However bromides are still commonly used with good results for the treatment of canine and feline epilepsy (Boothe 2002). Bromides have been demonstrated to have multiple mechanisms of anti-epileptic action in vitro. These include alteration of membrane excitability, increase of GABAergic inhibition, and extracellular pH effects (Meierkord 2000).

A published abstract indicated that L-thyroxine (T4) could induce larval metamorphosis of Haliotis rufescens (Carpazio-ltuarte 1993). More recently Fukazawa et al. have demonstrated that both T4 and T3 can induce larval metamorphosis in two different species of Haliotis (Fukazawa 2001). Hypothyroidism has been discovered in animals which are genetically prone to seizures (Ng 2001, Mills 1988). Additionally, hypothyroidism exacerbates canine epilepsy and may be an independent cause of seizures in dogs, these conditions are treated by thyroid hormone supplementation (www.canine-epilepsy.com, Boothe 2001). At least two known anti-epileptic drugs have been demonstrated to have receptor binding characteristics similar to thyroxine. Diphenylhydantoin and diazepam both competitively inhibit the binding of thyroxine to thyroxine-binding globulin (Camerman 1981). Hypothyroidism may also lead to seizures in humans (Bryce 1992, Selim 2001, Doherty 2001). Interestingly hyperthyroidism has been associated with seizures as well.

In order to eliminate the possibility that a very wide range of non-specific biological molecules and ions can induce abalone larval settlement, numerous substances have been studied. The following were not able to induce Haliotis rufenscens larvae settlement: serotonin, epinephrine, norepinephrine, histamine, acetylcholine, choline, n-butylamine, n-butyric acid, Na⁺, Mg⁺². Additionally, Cs⁺, Li⁺, choline⁺, and Tris⁺ were all found to be toxic to larvae at ≦9 mM (Baloun 1984). These substances are also thought to have little or no significant anti-epileptic effects.

These examples demonstrate the remarkable fact that several substances of various different chemical structure all have analogous effects upon abalone larval settlement and the prevention of seizures.
U.S. Pat. No. 5,675,061 to Powers et al. describes a method for the production of transgenic mollusks and the isolation/characterization of an abalone actin gene promoter region.
U.S. Pat. No. 5,902,732 to Hochman discloses a method for screening drug compounds for anti-epileptic activity and for determining cell viability inside polymeric tissue implants.
U.S. Pat. No. 6,271,197 to Berlin et al. relates to assays and reagents for identifying anti-fungal agents.
U.S. Pat. No. 6,566,580 to Van der Putten et al. describes the use of metabotropic glutamate receptor mGluR7 agonist for the facilitation of neurotransmitter release from a nerve ending and the treatment of neurological conditions, including epilepsy.
U.S. Pat. No. 6,727,082 to Berlin et al. relates to assays and reagents for identifying anti-fungal agents.
U.S. Pat. No. 6,994,977 to Gerard et al. describes a method for identifying inhibitors of C—C chemokine receptor 3.
U.S. Pat. No. 4,198,926 to Morse describes a method to induce spawning in shellfish.

### OTHER REFERENCES

Baloun, A. J., Morse, D. E., 1984. Ionic control of settlement and metamorphosis in larval Haliotis rufescens (Gastropoda). Biol. Bull. 167:124-138.
Barlow, L. A., 1990. Electrophysiological and behavioral responses of larvae of the red abalone (Haliotis rufescens) to settlement-inducing substances. Bull. Marine Sci. 46(2):537-554.
Bihi, R. I., Jefferys, J. G. R., and Vreugdenhil, M. 2003. The role of extracellular potassium in the epileptogenic transformation of recurrent GABAergic inhibition. Epilepsia 46 (S5):64-71.
Boothe, D. M. 2001. Small Animal Clinical Pharmacology and TherapeuticsW. B. Saunders, p. 625.
Boothe, D. M., George, K. L., and Couch, P. 2002. Disposition and clinical use of bromide in cats. J. Am. Vet. Med. Assoc. 221:1131-1135.
Bragin, A., Penttonen, M., and Buzsaki, G. 1997. Termination of epileptic afterdischarge in the hippocampus. The Journal of Neuroscience. 17(7): 2567-2579.
Bryce, G. M., and Poyner, F. 1992. Myxedema presenting with seizures. Postgraduate medical journal. 68(795): 35-36.
Camerman, A., and Camerman, N. 1981. On the crystallography and stereochemistry of antiepileptic drugs. Acta Cryst. B37: 1677-1679.
Carpizo-Ituarte, E., and Rosa-Velez, J. D. 1993. L-thyroxine induces metamorphosis in two species of marine gastropods. Amer. Zool. 1993; 33(5): 41A.
Doherty, C. 2001. Neurologic manifestations of thyroid disease. Neurologist 7(3):147-157.
Fisher, R. S. 1989. Animal models of the epilepsies. Brain Res. Rev. 14:245-278.
Fukazawa, H., Hirai, H., Hori, H., Roberts, R. D., Nukaya, H., Ishida, H., Tsuji, K., 2001. Induction of abalone larval metamorphosis by thyroid hormones. Fish. Sci. 67, 985-988.
Jones, O. T. 2002. Ca²⁺ channels and epilepsy. Eur. J Pharm. 447:211-225.
Kang, K. H., Kim, B. H., and Kim, J. M. 2004. Induction of larval settlement and metamorphosis of the abalone, Haliotis discus hannai larvae using bromomethane and potassium chloride. Aquacuture 230:249-259.
Lenzen, K. P., Heils, A., Lorenz, S., et al. 2005. Supportive evidence for an allelic association of the human KCNJ10 potassium channel gene with idiopathic generalized epilepsy. Epilepsy Research 63:113-118.
Meierkord, H., Grunig, F., Gutschmidt, U., et al. 2000. Sodium bromide: effects on different patterns of epileptiform activity, extracellular pH changes and GABAergic inhibition. Naunyn-Schm. Arch Pharm. 361:25-32.
Mills, S. A., and Savage, D. D. 1988. Evidence of hypothyroidism in the genetically epilepsy-prone rat. Epilepsy Res. 2, (2):102-10.
Morse, D. E., Hooker, N., Duncan, H., Hooker, N., Jensen, L. 1979. γ-Aminobutyric acid, a neurotransmitter, induces planktonic abalone larvae to settle and begin metamorphosis. Science 204: 407-410.
Morse, D. E., Duncan, H., Hooker, N., et al. 1980. GABA induces behavioral and developmental metamorphosis in planktonic molluscan larvae. Federation Proceedings. 39(14):3237-3241.
Ng, L., Pedraza, P. E., Faris, J. S. et al. 2001. Audiogenic seizure susceptibility in thyroid hormone receptor β-deficient mice. NeuroReport 12(11):2359-2362.
Perucca, E. 2005. An Introduction to Antiepileptic Drug. Epilepsia 46s4:31-37.
Roberts, R. 2001. A review of settlement cues for larval abalone (Haliotis spp.). J Shellfish Res. 20:571-786.
Searcy-Bernal, R., Salas-Garza, A. E., Flores-Aguilar, R. A., Hinojosa-Rivera, P. R. 1992. Simultaneous comparison of methods for settlement and metamorphosis induction in red abalone (Haliotis rufescens). Aquaculture 105: 241-250.
Selim, M. H. 2001. Neurologic aspects of thyroid disease. Neurologist 7(3):135-146.
Slattery, M. 1992. Larval settlement and juvenile survival in the red abalone (Haliotis rufescens): an explanation of inductive cues and substrate selection. Aquaculture 102:143-153.
Trapido-Rosenthal, H. G., Morse, D. E. 1985. L-a, N-diamino acids facilitate GABA induction of larval metamorphosis in a gastropod mollusc Haliotis rufescens. J Comp. Physiol. B. 155, 403-414.
Wallace, R. 2004. A potassium channel is associated with resistance to epilepsy. Epilepsy Currents 4(6) :245-247.
Yool, A. J., Grau, S. M., Hadfield, M. G., et al. 1986. Excess potassium induces larval metamorphosis in four marine invertibrate species. Biol. Bull. 170:255-266.

### SUMMARY OF THE INVENTION

It is therefore an object of the present invention to provide a drug screening assay

procedure using an abalone settlement assay.

It is another object of the invention to provide a means for realizing successful screening for anti-epileptic compounds.

In yet another objective of the present invention is to provide an assay that will facilitate the search for new anti-epileptic compounds.

A further objective of the present invention is to provide a method for better

understanding currently known substances.

In order to accomplish these and other objectives of the invention, a novel assay/method is provided utilizing marine organisms of the genus Haliotis implementing settlement induction in a novel test for the screening of anti-epileptic, anti-seizure, and anti-convulsant compounds/substances.

In accordance with the present invention the use of a mollusk for drug screening assays

can provide an economical/low cost, fast and sensitive method for determining potential anti-epileptic compounds without the use of costly or toxic chemicals.

### BRIEF DESCRIPTION OF THE DRAWING

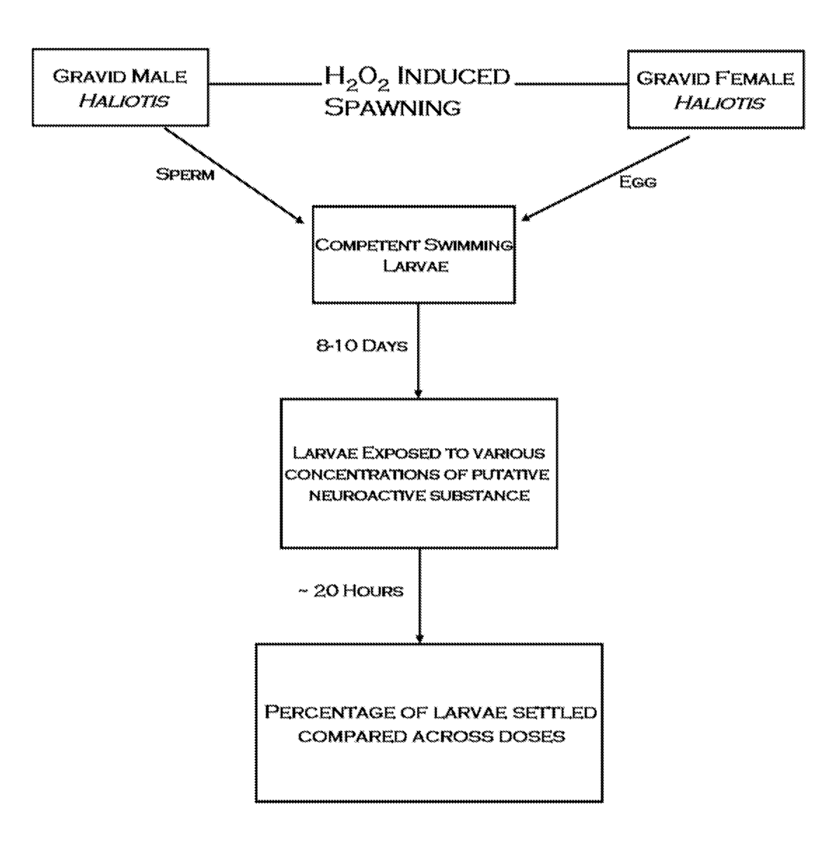

The foregoing and other objects, aspects and advantages of the present invention will be more clearly understood from the following detailed description taken in conjunction with the accompanying flow chart (FIG. 1). FIG. 1 illustrates a flow chart containing the preferred embodiment of the present process/invention.

### DETAILED DESCRIPTION OF THE INVENTION

### Problems Discovered by the Inventors

Current anti-convulsant drug screening assays primarily rely upon animal models using rats, and to a lesser extent mice and cats. These tests are relatively expensive, time consuming, and require the use of mammals. Additionally, these assays are not sensitive to all potential anti-epileptic compounds.

### Key Steps of the Invention

As outlined in FIG. 1 the present invention utilizes the information described above to develop a novel assay using Haliotis settlement induction as a test for anti-epileptic (anti-seizure, anti-convulsant) substances.

Gravid adult H. rufescens will be housed in aquaria. Male and female abalone will be selectively removed and placed in a separate aquarium where they will be induced to spawn using the hydrogen peroxide induction technique first described by Morse (U.S. Pat. No 4,198,926).

The competent swimming larvae will be maintained in fresh, filtered seawater with ultraviolet sterilization for 8-10 days post fertilization. Small glass vials will be filled with filtered sea water (15° C.) and approximately 150 swimming larvae. Various concentrations of the putative anti-epileptic substance will then be added to individual vials. Additionally, a control vial will have an equivalent volume of filtered sea water added. Penicillin G and dihydrostreptomycin (150 ppm) will be utilized in all vials. Twenty hours later the percent of larvae settled will be determined in a blinded fashion by visual inspection. The percent larva settled will then be compared across doses. The data obtained will be used to produce a dose-response curve to abalone larval settlement using the substance of interest.

The dose-response curve formed is then compared with various other assays and eventually the clinical effects in humans. Additionally, a natural extension of the current invention is the use of known abalone settlement inducers as novel anti-consulvants.

### Advantages of the Present Invention

A drug screening assay which uses abalone will be more economical. Several adult abalone may be kept in an aquarium requiring minimal manpower to maintain. These can provide many thousands of larvae. The assay itself is also less time consuming than many animal models of epilepsy and does not require the use of costly or toxic chemicals. Additionally, the use of a mollusk for an experimental assay raises fewer ethical concerns than mammals, which must be subjected to painful procedures and confinement. It is also possible that using an abalone settlement assay will provide information which is complementary to rather than entirely a replacement for current techniques.

While the present invention has been described and illustrated with respect to preferred embodiments, it is not intended to limit the invention, except as defined by the following claims. Furthermore, numerous modifications , changes, and improvements will occur to those skilled in the art without departing from the spirit and scope of the invention.

What is claimed is:

1. A method for screening drug candidate substances for neuroactive properties comprising:
(a) an aquarium housing adult marine mollusks;
(b) a separate aquarium for the induction of spawning and production of competent swimming larvae;
(c) the separation of swimming larvae at a critical time point post-fertilization into small glass vials;
(d) adding the drug candidate at various concentrations to the vials containing larvae; and the measurement of larval settlement induction at each concentration.

2. The method of claim 1 where the marine mollusk used is of the genus Haliotis.

3. The method of claim 1 where induction of spawning is by hydrogen peroxide with or without a catalyst.

4. A method using claim 1 pertaining to the screening for anti-epileptic/anti-seizure/anti-convulsant substances.

5. A method using claim 1 pertaining to the screening for substances which treat mental disease.

6. A method using claim 1 pertaining to the screening for substances useful for the therapy of neurological disease.

7. The method of claim 1 where the induction of spawning is by an oxidizing agent other than hydrogen peroxide.

8. The method of claim 1 where induction of spawning is by irradiation of water with ultra-violet light.

9. Any variation of the method of claim 1 which uses greater or fewer aquariums for mollusk housing.

10. The method of claim 1 where induction of spawning is by any method.

11. The method of claim 1 where induction of spawning is by thermal stimulation.

12. The method of claim 1 where induction of spawning is by the use of ozone.

13. The method of claim 1 where induction of spawning is by exposure to air.

14. The method of claim 1 where induction of spawning is by handling.

15. The method of claim 1 where induction of spawning is by electrical stimulation.

16. The method of claim 1 where the marine mollusk used is of a genus other than Haliotis.

17. Any variation of the method of claim 1 which still depends upon the measurement of the settlement of mollusk larvae.

18. The use of natural mollusk larva settlement cues as substances for pharmacological applications.
